(19) United States
(12) Patent Application Publication
(10) Pub. No.: US 2010/0311788 A1
(43) Pub. Date: Dec. 9, 2010

(54) Salts of Clopidogrel With Polyanions and Their Use for Manufacturing Pharmaceutical Formulations

(76) Inventors: Lars Dähne, Berlin (DE); Barbara Baude, Schwielowsee (DE); Maria Gonzalez Ferreiro, Berlin (DE)

Correspondence Address: Fanelli Strain & Haag PLLC, 1455 Pennsylvania Ave., N.W., suite 400, Washington, DC, 20004, US

(21) Appl. No.: 12/086,559

(22) PCT Filed: Dec. 15, 2006
(86) PCT No.: PCT/EP2006/012129

§ 371 Date: Aug. 24, 2010

(51) Int. Cl. A61K 31/4365; C07D 495/04; C08B 37/10; C07H 21/04; A61P 7/02; C08F 228/02; C08F 30/02

(52) U.S. Cl. 514/301; 546/114; 536/21; 536/23.1; 526/287; 526/274

(57) ABSTRACT

The invention relates to salts of the active ingredient clopidogrel with polyanions. The salts are predominantly in amorphous form, and the polyanions have at least 4 negative charges.

The present invention relates to salts of the active ingredient clopidogrel, and to methods for preparing them and their use in pharmaceutical formulations.

### BACKGROUND OF THE INVENTION

Clopidogrel (methyl (+)-(S)-α-(2-chlorophenyl)-6,7-dihydrothieno[3,2-c]pyridine-5(4)-acetate) (FIG. 1) is disclosed as active ingredient in EP-A-0 099 802 and U.S. Pat. No. 4,529,596. Clopidogrel acts as inhibitor of platelet aggregation and is therefore preferably employed for preventing thromboembolic events such as, for example, stroke or myocardial infarction.

It is proposed in EP-A-0 281 459 to employ clopidogrel in the form of its inorganic salts, specifically the crystalline hydrogen sulphate. This document also discloses further salts of clopidogrel, but they are described as in some cases amorphous and/or as hygroscopic and are therefore difficult to purify and hard to formulate for pharmaceutical purposes.

According to EP 1 480 985 B1, the clopidogrel hydrogen sulphate predominantly employed in pharmaceutical formulations has, because of the acidic proton, a high acid strength which results in a reduced compatibility with excipients. This patent, as well as WO 2004/072085 A2 and EP 1415993 A1, proposes the use for salt formation of monovalent organic alkyl- and arylsulphonic acids which form substantially crystalline compounds and can readily be purified. These salts are prepared in organic solvents such as dioxane, toluene etc. The crystallinity desired for purification is particularly pronounced when the salts comprise frequently adsorbed solvent residues as solvate.

The disadvantages of the crystalline clopidogrel hydrogen sulphate and of the crystalline organic alkyl- or arylsulphonates are a limited solubility and slow kinetics of dissolution. U.S. Pat. No. 6,284,277 and WO 2004/026879 therefore disclose preparation of an amorphous formulation of clopidogrel hydrogen sulphate having better solubility than that of the crystalline form by freeze drying the crystalline clopidogrel hydrogen sulphate in the presence of small molecules such as mannitol or alanine, or uncharged polymers (WO 2004/026879) such as polyvinylpyrrolidone, polyethylene glycol, hydroxypropylcellulose. However, the very acidic nature of the hydrogen sulphate remains, thus possibly having adverse effects on the excipients. For example, polyethylene glycol decomposes rapidly under acidic conditions.

### SUMMARY OF THE INVENTION

It has surprisingly been found in our investigations that, contrary to the disclosures in EP-A-0 281 459, WO 2004/072085 A2, EP 1415993 A1, EP 1 480 985 51, clopidogrel also forms with polyanions stable, substantially amorphous salts which are suitable for manufacturing oral pharmaceutical formulations of the active ingredient. The polyanions can be, for example, carrageenan, polystyrenesulphonate (PSS) or polyvinyl phosphate.

An embodiment of the present invention thus relates to salts of clopidogrel with polyanions. Another embodiment relates to the use of these salts in pharmaceutical formulations. A further embodiment also relates to stable mixed salts which can be precipitated from said clopidogrel-polyanion salts together with monovalent clopidogrel salts such as, for example, hydrochloride. The content of the polyanion salt in these compounds should be at least 10%, preferably at least 20%, more preferably at least 30%, and advantageously more than 40%. A further embodiment of the present invention additionally relates to the process for preparing clopidogrel-polyanion salts, preferably involving a precipitation from acidic aqueous solutions.

Preferably, the polyanion salts of clopidogrel are stable and non-hygroscopic. Further, the salts preferably have a high content of clopidogrel and a low content of additional hydrogen ions. Furthermore, the salts can be prepared on the industrial scale. Moreover, the salts typically dissolve easily and rapidly in the gastrointestinal tract. Particularly for pharmaceutical applications stable and non-hygroscopic polyanion salts of clopidogrel are preferred.

Clopidogrel can be employed both as racemic mixture of the two isomers and as pure isomer, with preference for the (S)-(±) isomer because of the higher activity.

Polyanions are macromolecular compounds having molecular weights preferably above 500 g/mol and at least 4 negative charge units, preferably more than 7 and very particularly preferably having more than 10 charges per molecule. The anionic charges are preferably achieved by acid groups having pKₐ values of <3.5, which are known for example for organic sulphate, sulphonate or phosphate groups and some carboxylate groups. Known polyanions having such properties are on the one hand naturally occurring compounds and modifications thereof, such as, for example, carrageenan, chondroitin sulphate, heparin, dextran sulphate, sulphoethylcellulose, DNA. Synthetic polyanions are additionally suitable, such as, for example, polystyrenesulphonate, polyanetholesulphonate, polyvinyl sulphate, polyvinylsulphonate, polyphosphate, polyvinylphosphonate. Salt formation with heparin, polyanetholesulphonate or with DNA allows combination products to be manufactured, with parenteral formulations (in some circumstances as depot product) being also possible, in contrast to oral uses of clopidogrel to date.

The prepared polysalts are, in contrast to the crystalline salts described to date, are partially amorphous and preferably substantially or predominantly amorphous or completely amorphous, allowing better kinetics of dissolution to be achieved than with the pure crystalline salts. Nevertheless, the polyanion salts can readily be purified and prepared in high purity, as described in the examples. The polyanion salts can also be prepared as mixed salts with monovalent anions such as, for example, hydrochloride, thus
1. achieving a stabilization of the inorganic salts such as, for example, of the hydrochloride,
2. achieving a higher content of active ingredient in the total composition,
3. the solubility decreasing only slightly while the substantially amorphous nature is retained.

The prepared polyanion salts comprise only few free H^{f}-ions in comparison to the clopidogrel hydrogen sulphate salt (HSO₄⁻), so that the acidity is distinctly less and harmful side effects on excipients or the body can be avoided.

It has surprisingly been found that the desired clopidogrel-polyanion salts can be easily precipitated when solutions of freely soluble salts of clopidogrel are added to aqueous polyanion solutions in the low pH range. The precipitates can easily be removed by centrifugation or filtration. The freely soluble clopidogrel salts used as starting material preferably have inorganic anions, like clopidogrel hydrogen sulphate, the hydrobromide or the particularly readily soluble hydrochloride. An additional or optional freeze drying step with an optional subsequent removal, for instance by washing, of the substituted anions can be performed.

These starting materials can also be prepared in situ from free clopidogrel base before the reaction with the polyanion, in particular by the following 3 methods:
a) Dissolving the resinous clopidogrel base in methanol, reacting with an HCl solution and removing the methanol by evaporation.
b) Dissolving the resinous clopidogrel base in HCl with dispersion of the clopidogrel drops
c) Extraction of the clopidogrel base into a water-immiscible organic solvent such as, for example, chloroform, diethyl ether or ethyl acetate and subsequent shaking with a solution of the appropriate polyanion in hydrochloric acid.

It has been found that, in contrast to the methods of preparation with monovalent anions, instead of equimolar ratios between clopidogrel and anion it is beneficial to use a distinct excess of clopidogrel. This on the one hand drastically increases the yield of precipitated salt, and on the other hand leads to a greater loading of the polymer and to a better clopidogrel/polyanion ratio.

The preparation of the clopidogrel-polyanion salts is described in some examples below and shown in the following figures.

### BRIEF DESCRIPTION OF THE DRAWINGS

FIG. 1: Structure of the active ingredient clopidogrel and reaction scheme for preparing a clopidogrel-polyanion salt

FIG. 2: Absorption spectra of clopidogrel hydrochloride in water at pH 1.2 in various concentrations (solid curve (2): 0.015M and dashed curve(1): 0.0015M)

FIG. 3: Plots of release of clopidogrel from the salts at 25° C. in 0.05M HCl (solid line), and the maximum value at complete dissolution (dashed line)
3a) release of clopidogrel from clopidogrel-PSS
3b) release of clopidogrel from clopidogrel-carrageenan
3c) release of clopidogrel from the clopidogrel-carrageenan/HCl mixed salt

FIG. 4: Powder diffractograms of the prepared clopidogrel-polyanion compounds

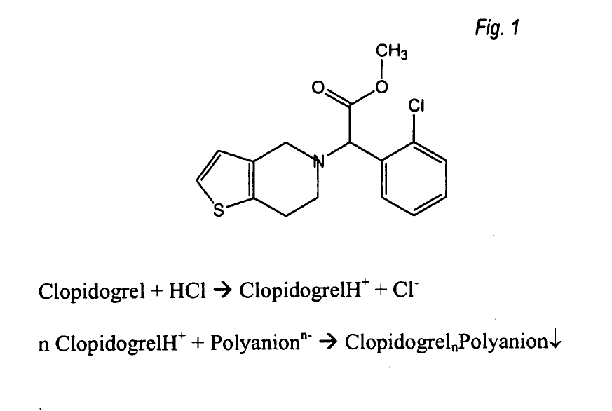

### DETAILED DESCRIPTION OF PREFERRED EMBODIMENTS

### Analytical Characterization of the Samples

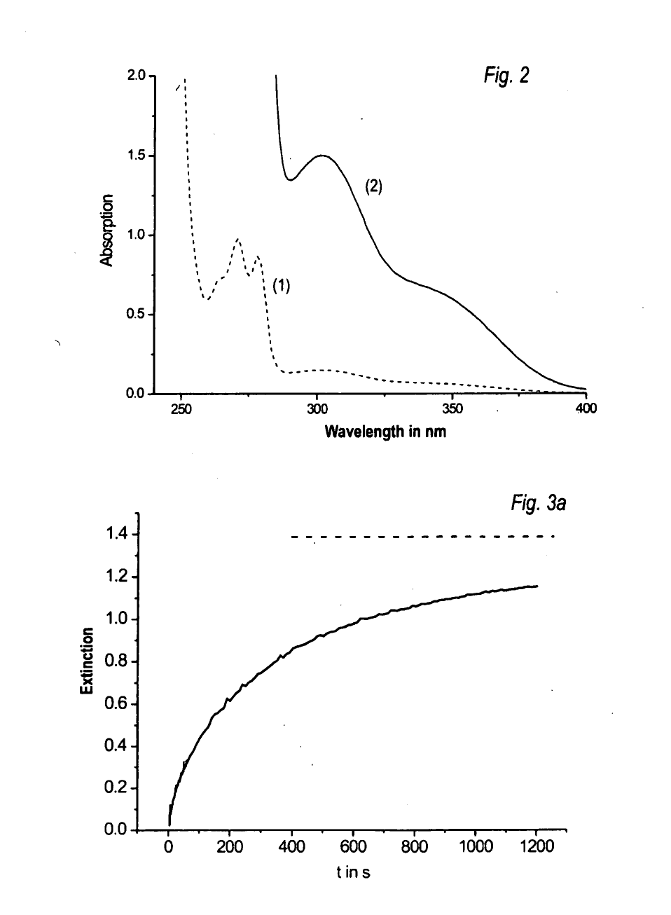

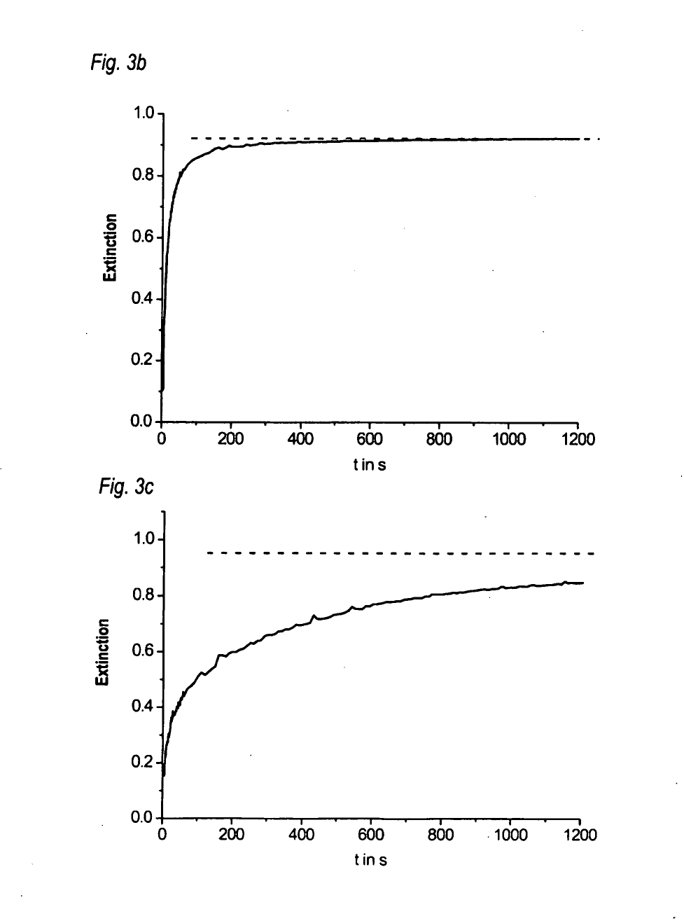

UV/Vis spectroscopy (Varian, Cary 50) was used for qualitative and quantitative characterization of the prepared compounds and provides a characteristics spectrum in the range 250-285 nm (FIG. 2) for clopidogrelli^{f}in water (the spectrum of the base in methanol differs therefrom), with characteristic UV/Vis peaks lying at

λₘₐₓ=271 nm ε=680 L/mol×cm
λₘₐₓ=278 nm ε=603 L/mol×cm
λₘₐₓ=301 nm ε=105 L/mol×cm
λₘₐₓ=342 nm ε=46 L/mol×cm.

The ratio between clopidogrel and polyanion in the prepared compounds was further ascertained by CHN analyses. The kinetics of dissolution of the clopidogrel-polyanion salts and the solubilities were determined in 0.05M HCl by UV/Vis spectroscopy. The X-ray investigations on the crystalline/amorphous ratio were carried out on powder samples with an STOE STADI P transmission diffractometer, Cu K alpha 1 radiation (1.5406 A).

### Example 1

### Preparation of Clopidogrel-PSS from ClopidogrelHBr

804 mg (2 mmol) of clopidogrelHBr are dissolved in 20 mL of 0.2M HCl. 10 ml of a 0.1 monomolar solution (molarity based on monomers) of the sodium salt of polystyrenesulphonic acid (PSS) are added to this solution while stirring. The solution is cooled to 5° C. while stirring, and the precipitate is then removed by centrifugation. The precipitate is washed 3 times with 1 mL of 0.05M HCl each time and then dried in vacuo.

Yield: theor: 505 mg, found 230 mg=46%

Active ingredient loading: The active ingredient loading was analysed by dissolving 1.77 mg of product in 2 mL of 0.05M HCl (0.885 g/L) and recording the UV/Vis spectrum. The extinction at 271 nm is E=1.628, being composed of the extinction of PSS ε₂₇₁=330 L/mol×cm and the extinction of clopidogrel ε₂₇₁=680 L/mol×cm to give ε₂₇₁=1010 L/mol×cm. This results in a first approximation (M_{w}=505 g/mol for 1:1 product) in a loading of 58.5% m/m of clopidogrel in the product, which is somewhat below the theoretically achievable 63%. 57.9% were found in a second analysis. This means that 92% of the PSS groups are loaded with clopidogrel.

Elemental analysis: Elemental analysis of the starting materials and of the prepared compound yields the values listed in Table 1, using the theoretical PSS composition for calculating the compound and assuming a 1:1 loading ratio between PSS and clopidogrel in the product composition:

TABLE 1
Percentage distribution of the elements in the
starting materials and the prepared compound
Molecular weights: Clopidogrel hydrobromide 402 g/mol, PSS
(monomer unit) 206 g/mol, clopidogrel-PSS 505.45 g/mol

| | | Clopidogrel | | | |
|---|---|---|---|---|---|
| | Clopidogrel hydro- | hydro- | | Clopidogrel | Clopidogrel |
| | bromide | bromide | Na PSS | PSS | PSS |
| Compound | % | % | % | % | % |
| Element | theor. | found | theor. | theor. | found |
| C | 47.71 | 47.17 | 46.60 | 56.98 | 54.64 |
| H | 4.22 | 4.365 | 3.40 | 4.75 | 5.028 |
| N | 3.48 | 3.463 | 0.00 | 2.77 | 2.773 |
| O | 7.95 | — | 23.30 | 15.83 | — |
| S | 7.95 | 7.981 | 15.53 | 12.66 | 12.31 |
| Cl | 8.81 | 8.614 | 0.00 | 7.01 | 7.4 |
| Br | 19.88 | 19.4 | 0.00 | 0.00 | — |
| Na | 0 | — | 11.17 | 0.00 | — |
| | 100.00 | 91 | 100.00 | 100.00 | 82.15 |

The elemental analyses confirm the composition of the desired product clopidogrel-PSS. Likewise, the spectroscopic result of a somewhat lower loading is verified, because the carbon content and sulphur content are slightly lower than would be expected with 100% clopidogrel loading.

### Solubility

The solubility of the compounds was determined by over-layering in each case 20 mg with 500 μL of 0.05M HCl and incubating at 25° C. with shaking for 48 h. The precipitate was spun down thoroughly, and the supernatant was diluted in 0.05M HCl and the spectrum was recorded. The solubility L in g/L of the compound was determined from the extinction. L_{clopiPSS}=0.0195 mol/L=9.9 g/L

### Rate of Dissolution

The kinetics of dissolution were measured in 0.05M HCl solution (pH 1.3) at 25° C. For this purpose, a defined amount of the clopidogrel salt which had been powdered in a mortar was weighed into a 4 mL quartz cuvette and placed with a magnetic stirrer in the UV/Vis spectrometer. At time t₀=0 s, 2 mL of 0.05M HCl were added, and the increase in extinction at 271 nm was followed. The extinction when dissolution was complete at t_{∞} was determined after stirring for 20 h and briefly heating the sample at 50° C. FIG. 3a shows the kinetics of dissolution and the value when dissolution is complete (dashed line). Release after 20 min at 25° C. is 83% for the clopidogrel-PSS compound.

### Example 2

### Preparation of Clopidogrel-Carrageenan from Clopidogrel Base (Method (a), See Above)

6.42 g of clopidogrel (20 mmol) are dissolved in 100 Ml of methanol at room temperature. 200 mL of 0.2M HCl (40 mmol) are added thereto. The clear solution was concentrated to about 120 mL in a rotary evaporator and made up again to 150 mL with water. A clear 0.133M clopidogrel hydrochloride solution with a pH of 0.67 is obtained. 90 ml of this solution (12 mmol) were added to 60 mL of a 0.1M solution (6 mmol) of λ-carrageenan of type IV (Sigma-Aldrich) while stirring vigorously at room temperature. The solution is cooled while stirring to 5° C., and the precipitate is filtered off. The precipitate is washed 3 times by being resuspended each time in 24 mL of 0.05M HCl and filtered. The precipitate was then dried in vacuo.

Yield: theor: 3324 mg, found 2042 mg=61.4%

Active ingredient loading: The active ingredient loading was determined in duplicate in analogy to Example 1 and resulted in 57.26 and 57.19% respectively, i.e. 98% of the theoretical loading of 57.9%.

Elemental analysis: Elemental analysis of the starting materials and of the prepared compound results in the values listed in Table 2. The values measured for carrageenan differ distinctly from the values resulting from the formula. The measured carrageenan composition was used to calculate the theoretical values for the clopidogrel-carrageenan salt. The product composition was assumed to be a 1:1 charge ratio between carrageenan and clopidogrel.

TABLE 2
Percentage distribution of the elements in the
starting materials and the prepared compound
Molecular weights: Clopidogrel base 321 g/mol, carrageenan
(monomer unit) 256 g/mol, clopidogrel-carrageenan 554 g/mol

| | | Carrageenan 2 | Carrageenan 3 | | | |
|---|---|---|---|---|---|---|
| | | sulphate | sulphate | | | |
| | | groups | groups | | | |
| | | per | per | | | |
| | | galacto- | galacto- | | | |
| | Clopidogrel | pyranose | pyranose | | Clopidogrel | Clopidogrel |
| | base | dimer | dimer | Carrageenan | carra | carra |
| Compound | % | % | % | % | % | % |
| Element | theor. | theor. | theor. | measured | theor. | found |
| C | 47.71 | 28.24 | 22.86 | 22.28 | 44.46809 | 42.12 |
| H | 4.22 | 3.14 | 2.70 | 3.799 | 4.787874 | 4.796 |
| N | 3.48 | 0.00 | 0.00 | 0.145 | 2.560156 | 2.669 |
| O | 7.95 | 47.06 | 48.25 | — | — | — |
| S | 7.95 | 12.55 | 15.24 | 11.74 | 11.14950 | 11.75 |
| Cl | 8.81 | 0.00 | 0.00 | 0.3 | 6.291723 | 8.23 |
| Br | 19.88 | 0.00 | 0.00 | — | 0 | — |
| Na | 0 | 9.02 | 10.95 | — | 0 | — |
| Total | 100.00 | 100.00 | 100.00 | 38.26 | 64.37 | 69.6 |

The elemental analyses differ somewhat from the expected composition, possibly explicable by the non-uniform composition of the natural product carrageenan.

### Solubility

The solubility was determined in analogy to Example 1 and is in 0.05M HCl at 25° C.

L_{clopicarra}=0.0634 mol/L=35.1 g/L

### Rate of Dissolution

The kinetics of dissolution were determined in analogy to Example 1. FIG. 3b shows the kinetics of dissolution and the value when dissolution is complete (dashed line). Release after 20 min at 25° C. is 99% for the clopidogrel-carrageenan compound. The product dissolves very rapidly and dissolution is virtually 100% complete after only a few minutes.

### Example 3

### Preparation of a Clopidogrel-Carrageenan/HCl Mixed Salt from Clopidogrel Base (Method (b), See Above)

3.21 g of clopidogrel base (10 mmol) are dissolved in 50 mL of 2M HCl (100 mmol) by vigorous stirring and ultrasound. This is possible, in contrast to HCl of low concentration, within less than one minute. The solution is then adjusted to a pH of about 0 with 4 mL of 10M NaOH (90 mmol) and, while stirring vigorously, added to 100 mL of 0.05M aqueous λ-carrageenan solution (5 mmol). The pH is raised to pH 1 by adding 10M NaOH, and the solution is cooled to 5° C. while stirring. The precipitate is spun down, washed 3 times with 12 mL of 0.05M HCl each time and then dried in vacuo.

Yield: theor: 2770 mg, found 1620 mg=58.5%

Active ingredient loading: The active ingredient loading was determined in duplicate in analogy to Example 1 and the duplicate determination resulted in 72.26 and 74.1%, respectively, i.e. 126% of the theoretical value for the pure clopidogrel-carrageenan salt of 57.9% loading. The difference results from the formation of a mixed salt with clopidogrel hydrochloride as shown by the elemental analyses hereinafter. Pure clopidogrel hydrochloride could not be isolated on its own from aqueous solution and is described in the literature as being very hygroscopic. Calculation of the ratio between carrageenan anion and chloride anion from the loading (100% carrageenan salt 57.9% loading; 100% chloride salt=89.9% loading) results in a carrageenan:chloride ratio of 1.13.

Elemental analysis: Elemental analysis of the prepared compound provided the proof of a mixed salt. The carrageenan composition determined by the elemental analysis was used to calculate the theoretical values for the clopidogrel-carrageenan salt. The product composition was assumed to be a 1:1 charge ratio between carrageenan and clopidogrel (Table 3).

TABLE 3
Percentage distribution of the elements in the
prepared compound

Compound

| | Clopidogrel | Clopidogrel |
|---|---|---|
| | carra | carra |
| | % | % |
| Element | theor. | found |
| C | 44.46809 | 40.25 |
| H | 4.787874 | 4.381 |
| N | 2.560156 | 2.402 |
| O | — | — |
| S | 11.14950 | 11.4 |
| Cl | 6.291723 | 11.11 |
| Br | 0 | — |
| Na | 0 | — |
| Total | 64.37 | 69.54 |

The elemental analyses differ distinctly from the expected composition, resulting from the formation of the chloride mixed salt. In particular the high value for chlorine and the ratio between the carbon content and the chlorine content confirm the formation of the mixed salt, the latter specifically agreeing well with the ratio between carrageenan anion and chloride anion derived from the loading.

### Solubility

The solubility was determined in analogy to Example 1. In 0.05M HCl at 25° C. it is L_{clopicarra/HCl}=0.026 mol/L=14.4 g/L. This is distinctly lower than the solubility of the pure single salts, thus explaining the preferential formation of this mixed salt at low pH.

### Rate of Dissolution

The kinetics of dissolution were determined in analogy to Example 1. FIG. 3c shows the kinetics of dissolution and the value when dissolution is complete (dashed line). The clopidogrel-carrageenan/HCl mixed salt dissolves substantially less well than the pure carrageenan salt. Release after 20 min at 25° C. is only 89.5%.

### Example 4

### Preparation of Clopidogrel-Dextran Sulphate from Clopidogrel Hydrogen Sulphate (Method c)

0.418 g of clopidogrel hydrogen sulphate (1 mmol) is dissolved in 30 ml of water and covered in a separating funnel with a layer of a mixture of 50% ethyl acetate and 50% diethyl ether. 3.5 mL of 10M NaOH are added thereto and shaken for a lengthy period. After the initially cloudy aqueous phase has become substantially clear, it is separated off. The organic phase is washed once with 20 mL of water. Then 5 mL of a 0.1M dextran sulphate solution and 5 mL of a 0.3M HCl are added and shaken for a lengthy period. A white precipitate separates out in the aqueous phase. After removal of the organic phase, the precipitate is spun down, washed 3 times with 0.05M HCl and then dried in vacuo.

Molecular weight: M_{w}=617 g/mol

Yield: theor: 308.5 mg, found 212 mg=68.7%

Active ingredient loading: The active ingredient loading was determined in duplicate in analogy to Example 1, and the duplicate determination resulted in 59.2 and 60.7%, respectively. This is somewhat more than would have been expected from the sulphate content indicated by the manufacturer (Sigma). Only the solubilities and the percentage clopidogrel content were determined for this and the following compounds.

### Solubility

The solubility was determined in analogy to Example 1. In 0.05M HCl at 25° C. it is L_{clopidextran sulphate}=0.019 mol/L=11.7 g/L. This is distinctly lower than the solubility of the pure single salts, thus explaining the preferential formation of this mixed salt at low pH.

### Example 5

### Preparation of Clopidogrel-Heparin from Clopidogrel Base (Method a)

The salt (M_{w}=584 g/mol) was prepared in analogy to Example 2 in a smaller batch. The yield was 22%, the active ingredient loading was determined to be 53.4%, almost corresponding to the theoretical value (54.9%). The solubility in 0.05M HCl is L_{clopihaparin}=0.084 mol/L=49.1 g/L.

### Example 6

### Preparation of Clopidogrel-Chondroitin Sulphate from Clopidogrel Base (Method a)

The salt was (M_{w}=530 g/mol) prepared in analogy to Example 2 in a smaller batch. However, no precipitate separated out in this case. The solution was therefore freeze dried and the NaCl was removed from the resulting solid by washing 3 times. The yield was 15%, the active ingredient loading was determined to be 51.7% (theoretical 60.6%). The low active ingredient loading is perhaps explained by the extremely high solubility of L_{clopichondroitin sulphate}=0.45 mol/L=238 g/L.

### Example 7

### Preparation of Clopidogrel-Polyvinyl Phosphate from Clopidogrel Base (Method a)

The salt (M_{w}=443 g/mol) was prepared in analogy to Example 6. A gelatinous composition was obtained, but a powder also resulted after freeze drying. The yield was 35%, the active ingredient loading was determined to be 73.2% (theoretical 72.5% for one clopidogrel per phosphate group). The solubility is L_{clopipolyvinyl phosphate}=0.22 mol/L=97 g/L, with insoluble particles remaining even at high dilution.

### Example 8

### Preparation of Clopidogrel-Polyvinylsulphonate from Clopidogrel Base (Method a)

The salt (M_{w}=428 g/mol) was prepared in analogy to Example 6. The yield was 19%, the active ingredient loading was determined to be 48% (theoretical 75%). The low active ingredient loading is perhaps explained by the extremely high solubility of the salt with L_{clopipolyvinylsulphonate}=0.4 mol/L=171 g/L and possibly by the low hydrophobicity of the polyanion.

### Analysis of the Crystallinity of the Compounds

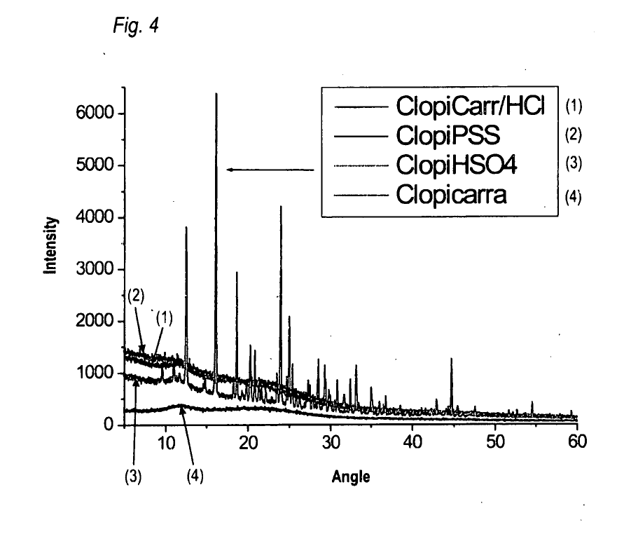

Measurements were carried out in an X-ray powder diffractometer on the samples from Examples 1-3, clopidogrel-PSS, clopidogrel-carrageenan, clopidogrel-carrageenan/HCl and, for comparison, clopidogrel hydrogen sulphate. FIG. 4 shows the corresponding spectra. Whereas clopidogrel hydrogen sulphate (green; curve (3)) shows a substantially crystalline structure, the structure to be found with all the prepared products is substantially amorphous. There are still slight gradations between the clopidogrel-PSS (red in the figure; curve (2)) with a completely amorphous nature and the clopidogrel-carrageenan (curve (4)) with a quite weak structure and the clopidogrel-carrageenan/HCl (curve (1)) with a somewhat more pronounced structure, but still substantially amorphous.

What is claimed is:

1. A salt of clopidogrel with a polyanion, wherein the salt is substantially in amorphous form, said polyanion having at least 4 negative charges.

2. The salt according to claim 1 wherein the polyanion is an organic polyanion comprising charge carriers selected from the group consisting of sulphate, sulphonate, or phosphate groups.

3. The salt according to claim 1, wherein the polyanion is selected from the group consisting of carrageenans, chondroitin sulphate, alginate sulphate, dextran sulphate, sulphoethylcellulose, and heparin.

4. The salt according to claim 1, wherein the polyanion comprises a synthetic polymer having charge carriers selected from the group consisting of sulphate, sulphonate, phosphate, or phosphonate groups.

5. The salt according to claim 1 wherein the polyanion is selected from the group consisting of heparin or DNA, combination products.

6. A salt according to claim 1 further comprising a monovalent clopidogrel salt, wherein the polyanion salt content is at least 10%.

7. A method for preparing a salt according to claim 1, wherein the salt is precipitated from a predominantly aqueous solution of a freely soluble clopidogrel salt with an aqueous solution of the polyanion.

8. (canceled)

9. A pharmaceutical formulation comprising the salt according to claim 1 and a pharmaceutically acceptable carrier or excipient.

10. The salt according to claim 1, wherein the polyanion has at least 7 negative charges.

11. The salt according to claim 1, wherein the polyanion has at least 10 negative charges.

12. The salt according to claim 6, wherein the polyanion salt content is at least 30%.

13. The salt according to claim 4, wherein the synthetic polymer is selected from the group consisting of polystyrenesulphonate, polyanetholesulphonate, polyvinyl sulphate, and polyvinyl phosphate.
